(19) United States
(12) Patent Application Publication
(10) Pub. No.: US 2016/0192908 A1
(43) Pub. Date: Jul. 7, 2016

(54) SUBJECT INFORMATION ACQUIRING DEVICE, SUBJECT INFORMATION ACQUIRING METHOD, AND PROGRAM

(71) Applicant: CANON KABUSHIKI KAISHA, Tokyo (JP)

(72) Inventors: Shigeaki Okumura, Kyoto-shi (JP); Hirofumi Taki, Kyoto-shi (JP); Toru Sato, Kyoto-shi (JP); Aya Kita, Kobe-shi (JP); Kenichi Nagae, Yokohama-shi (JP)

(21) Appl. No.: 14/904,833

(22) PCT Filed: Jul. 14, 2014
(86) PCT No.: PCT/JP2014/069126

§ 371 Date: Jan. 13, 2016

(30) Foreign Application Priority Data
Jul. 19, 2013 (JP) 2013-150761

(51) Int. Cl. A61B 8/08; A61B 8/00; G01S 7/52

(52) U.S. Cl. CPC A61B 8/5276; G01S 7/52085; A61B 8/5223; A61B 8/4254

(57) ABSTRACT

A subject information acquiring device includes a plurality of conversion element and a processing unit. Each of the plurality of conversion elements transmits acoustic waves as to a subject, receives reflected waves which have reflected within the subject, and converts the received reflected waves into time-sequence reception signals. The processing unit acquires information of within the subject by performing adaptive beam forming processing using a plurality of the reception signals. To output a plurality of times worth of reception signals based on each of a plurality of times of acoustic wave transmission, at least a part of the plurality of conversion elements perform acoustic wave transmission as to a predetermined region within the subject the plurality of times. In the adaptive beam forming processing, the processing unit performs integration processing of a plurality of correlation matrices obtained using the plurality of times worth of reception signals.

### TECHNICAL FIELD

The present invention relates to a subject information acquiring device, subject information acquiring method, and program. More particularly, the present invention relates to a technology to transmit acoustic waves to a subject, receive reflected waves which have reflected within the subject, and thus acquire information of within the subject.

### BACKGROUND ART

Ultrasound diagnosis devices are widely used as subject information acquiring devices in medical practice. Not only is form information reflecting difference in acoustic impedance within an organism obtained by ultrasound diagnosis devices; movement information of an object such as flow rate information of blood can also be obtained. In a pulse-Doppler system, phase change is detected in reflected waveforms from the object, and the movement speed is calculated. For example, in a case of measuring the flow rate of blood as movement information of the object, the phase change is detected of the received signals from the waves reflected from the blood cells within the blood vessels. There is the need to separate the reflected waves from the blood cells, and reflected waves from other objects in the vicinity other than the object to be measured, such as blood vessel walls, and so forth (also called “clutter component”).

PTL 1 describes separating the flow of the blood and the movement of walls of blood vessels, heart walls, and so forth which are clutter components, using a moving target indicator (MTI) filter, when calculating blood flow rate using the pulse-Doppler method. Three types of transmission intervals are provided for ultrasonic pulses, and the difference between the reception signal of the third pulse and the reception signal of the first pulse (first difference signal), the difference between the reception signal of the fourth pulse and the reception signal of the second pulse (second difference signal), and the difference between the reception signal of the fifth pulse and the reception signal of the third pulse (third difference signal), are each obtained. The phase difference between the first and second difference signals, and the phase difference between the second and third difference signals are obtained, and moreover the difference between the two phase differences is obtained.

### CITATION LIST

### Patent Literature

PTL 1 Japanese Patent Laid-Open No. 1-153144

### SUMMARY OF INVENTION

### Technical Problem

Phase change is detected in signals of waves reflected from blood cells when measuring the blood flow rate as described above, but the intensity of reflected waves from the blood cells is weak compared to reflected waves from blood vessel walls and the like in the periphery. Detecting phase change of the received signals in this state where unnecessary clutter component signals (clutter signals) from reflectors other than the object is included readily causes error. While PTL 1 describes a method using an MTI filter, there are cases where removal of clutter signals by such a filter is insufficient. Particularly, in cases where the clutter signals are great, or cases where reflectors other than the object are fluctuating (moving) due to pulsation of tissue near the blood vessel or shaking of the technician's hands or the like, accurately calculating the blood flow rate is difficult.

Noise due to clutter component is not restricted to cases of obtaining movement information of objects such as blood flow rate and the like, using the pulse-Doppler method as described above; noise is caused by the clutter component when generating an acoustic property distribution such as a common B-mode image, or the like, as well. This noise may cause deterioration in image quality of the acoustic property distribution.

It has been found to be desirable to suppress deterioration in acquisition accuracy of movement information and deterioration in image quality of acoustic property distribution, even in cases where unnecessary reflected waves exist besides reflected waves from the object.

### Solution to Problem

A subject information acquiring device according to the present invention includes: a plurality of conversion elements configured to each transmit acoustic waves as to a subject, receive reflected waves which have reflected within the subject, and convert the received reflected waves into time-sequence reception signals; and a processing unit configured to acquire information of within the subject by performing adaptive beam forming processing using a plurality of the reception signals. At least a part of the plurality of conversion elements perform acoustic wave transmission as to a predetermined region within the subject a plurality of times, thereby outputting a plurality of times worth of reception signals based on each of the plurality of times of acoustic wave transmission. In the adaptive beam forming processing, the processing unit performs integration processing of a plurality of correlation matrices obtained using the plurality of times worth of reception signals.

In a subject information acquiring method according to the present invention, time-sequence reception signals, output from a plurality of conversion elements which receive reflected waves which have reflected within a subject, are used to acquire information of within the subject. The method includes: a step to cause at least a part of the plurality of conversion elements to perform acoustic wave transmission as to a predetermined region within the subject a plurality of times; and a step to perform adaptive beam forming processing using the plurality of times worth of reception signals obtained by the plurality of times of acoustic wave transmission. In the adaptive beam forming processing, integration processing is performed of a plurality of correlation matrices obtained using the plurality of times worth of reception signals.

Further features of the present invention will become apparent from the following description of exemplary embodiments with reference to the attached drawings.

### BRIEF DESCRIPTION OF DRAWINGS

FIG. 1 is a schematic diagram of a subject information acquiring device according to a first embodiment.

FIG. 2 is a flowchart illustrating processing according to the first embodiment.

FIGS. 3A and 3B are schematic diagrams illustrating difference processing blocks.

FIG. 4 is a schematic diagram for describing processing at an adaptive signal processing block.

FIG. 5 is a schematic diagram illustrating an example of display.

FIG. 6 is a schematic diagram for describing a model for verifying effectiveness.

FIG. 7 is a diagram for describing the advantages of the first embodiment.

FIG. 8 is a schematic diagram of a subject information acquiring device according to a second embodiment.

FIG. 9 is a schematic diagram for describing a region where an image is obtained according to a third embodiment.

FIGS. 10A through 10C are schematic diagram illustrating transmission methods according to the third embodiment.

FIG. 11 is a flowchart for describing processing according to a fourth embodiment.

### DESCRIPTION OF EMBODIMENTS

Embodiments of the present invention will now be described with reference to the drawings. The same components will be denoted by the same reference numerals as a rule, and redundant description thereof will be omitted.

Note that in the present invention, the term “acoustic waves” typically means ultrasound, and includes elastic waves known as sound waves and ultrasound. The subject information acquiring device according to the present invention includes devices which transmit acoustic waves to a subject, receive reflected waves which have reflected within the subject (reverberated acoustic waves), and thus acquire information and numerical values of within the subject as data. Information within the subject that is obtained includes movement information of an object, information reflecting difference in acoustic impedance of tissue, and so forth.

### First Embodiment

The device configuration and processing flow of a first embodiment will be described. Description will be made regarding obtaining movement information of an object such as blood flow or the like, as information of within the subject.

### Configuration of Subject Information Acquiring Device

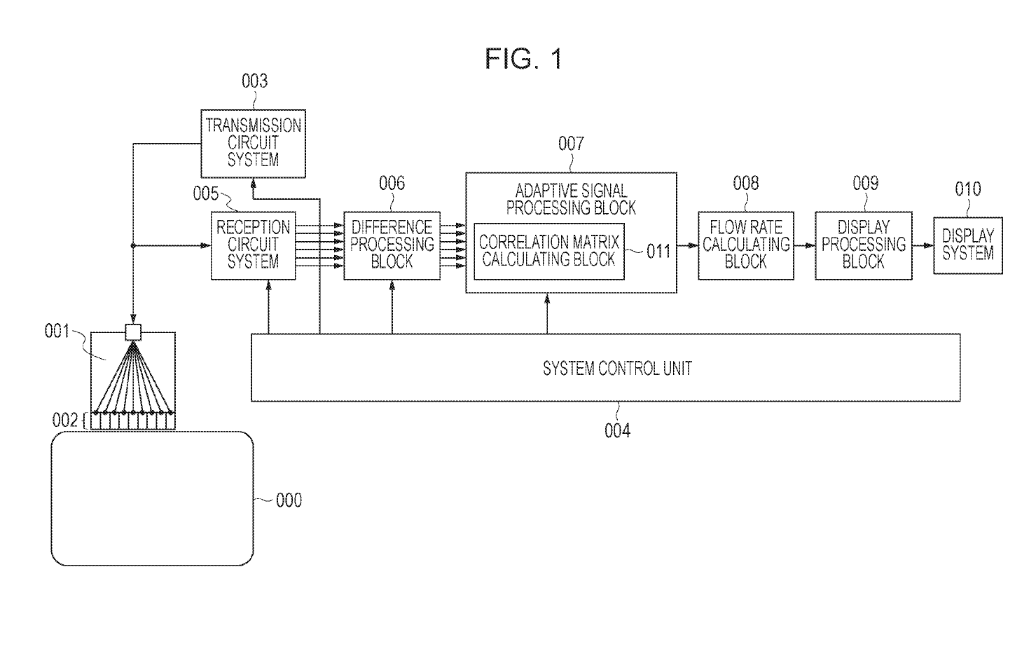

FIG. 1 is a schematic diagram illustrating the configuration of a subject information acquiring device according to the first embodiment. The subject information acquiring device includes a probe 001 having multiple conversion elements 002, a reception circuit system 005, a transmission circuit system 003, a difference processing block 006, an adaptive signal processing block 007, a flow rate calculating block 008, a system control unit 004, a display processing block 009, and a display system 010.

The probe 001 is a transmitter/receiver which transmits acoustic waves to a subject 000, and receives reflected waves which have reflected at multiple locations within the subject. The probe 001 has multiple conversion elements 002 which convert acoustic waves into electric signals (time-sequence reception signals). Though not illustrated in FIG. 1, the subject 000 includes therein an object (tissue having an acoustic impedance distribution, for example), and reflected waves from the object are received at the multiple conversion elements 002. The conversion elements 002 may be any sort of conversion elements as long as acoustic waves can be received and converted into electric signals. Examples include piezoelectric type conversion elements which use the piezoelectric phenomenon, conversion elements which use resonance of light, conversion elements which use change in electrostatic capacitance such as capacitive micromachined ultrasonic transducers (CMUT), or the like. The multiple conversion elements are preferably arrayed as a 1D array, 1.5D array, 1.75D array, or a 2D array.

The transmission circuit system 003 is a transmission signal generating circuit which generates voltage waveform transmission signals (pulse signals) having delay time and amplitude in accordance with a position of interest or direction of interest, under control signals from the system control unit 004. The transmission signals are each input to the conversion elements 002, and acoustic waves are transmitted as acoustic pulses (pulse waves) from the conversion elements 002. The reflected waves from the subject are received by the multiple conversion elements 002, and the reception signals output from each of the multiple conversion elements 002 are input to the reception circuit system 005.

The reception circuit system 005 is a reception signal processing circuit which amplifies the reception signals output in time sequence from the conversion elements 002, and converts into digital signals (digitized reception signals), and is configured including an amplifier, A/D converter, and so forth. One conversion element 002 receives a reflection wave based on one acoustic wave transmission, and a time-sequence reception signal is output the conversion element 002; this time-sequence reception signal is handled as one reception signal. In a case of receiving acoustic waves using M conversion elements 002, M reception signals, equal to the number M of conversion elements 002, are received for one acoustic wave transmission. Also, when looking at one conversion element 002, performing acoustic wave transmission N times yields N times worth of reception signals (i.e., N time-sequence reception signals) for the one conversion element 002. N and M are positive integers. Note that not only the analog reception signals output from the conversion elements 002, but also signals after processing such as amplification and digital conversion will also be referred to as reception signals.

Note that not all of the conversion elements 002 within the probe 001 have to be used when performing one acoustic wave transmission in the present embodiment. An arrangement may be made where one acoustic wave transmission (transmission beam forming) is performed using a part of the conversion elements 002 (a conversion element group) of the probe 001. In this case, acoustic waves can be transmitted over a wide range by repeating acoustic wave transmission while sequentially switching the conversion element groups to perform transmission. Also, at least part of the conversion elements 002 transmit acoustic waves to a predetermined region in the subject multiple times. The conversion elements 002 used for reception output reception signals for each acoustic wave transmission. That is to say, the conversion elements 002 output multiple times worth of reception signals.

The reception signals output from each output channel of the reception circuit system 005 are input to the difference processing block 006. The output channels of the reception circuit system 005 and the output channels of the multiple conversion elements 002 correspond. It should be noted, however, that one acoustic wave transmission may be performed using only a part of the conversion elements 002, so the number of conversion elements 002 and the number of output channels may not necessarily be the same number. In other words, there may be fewer output channels than conversion elements 002. For example, in a case of performing beam forming using every 32 conversion elements 002 out of 256 conversion elements 002, it is sufficient for the number of output channels to be 32.

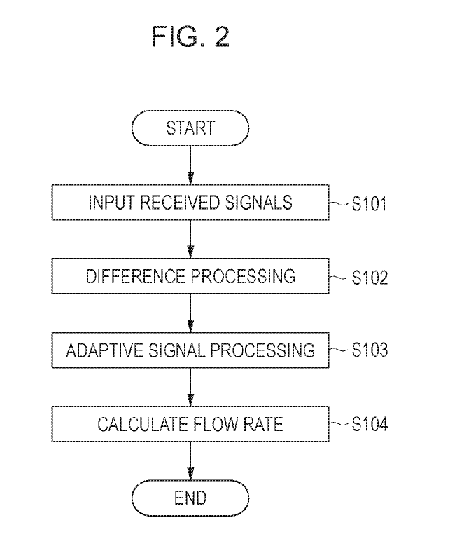

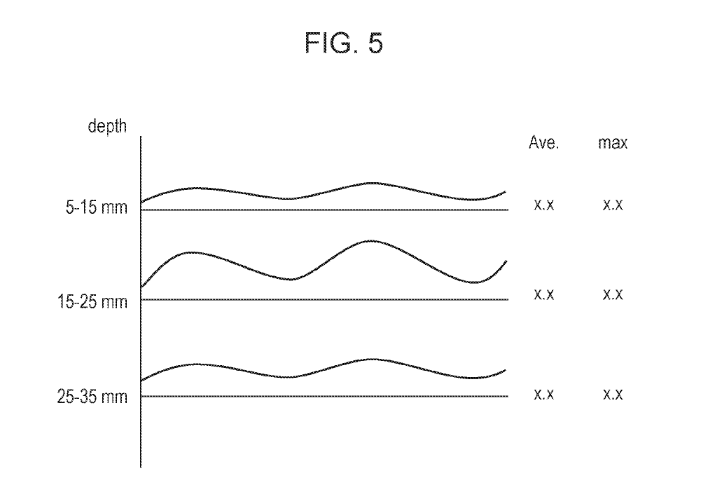

The difference processing block 006 is an extracting unit which extracts temporal variation components among the reception signals. Typically, the difference processing block 006 is configured using a difference filter such as an MTI filter. The difference processing block 006 uses the reception signals for several times, obtained based on several acoustic wave transmissions to the predetermined region for each output channel, to obtain multiple differences among the reception signals, as difference signals. The reception signals among which difference processing is performed are typically based on reflection waves of acoustic waves transmitted to the same region. Note however, that the term “same region” is not restricted to being the same region in the strictest sense, and includes a range regarding which there is no problem in performing processing deeming to be the same region. Details of the processing performed at the difference processing block 006 will be described in S102 in FIG. 2 and in FIG. 5.

The adaptive signal processing block 007 receives input of multiple difference signals, which are the differences among the reception signals output from the difference processing block 006. The difference processing block 006 according to the present embodiment is an adaptive signal processing unit which performs adaptive signal processing, and includes a correlation matrix calculating block 011.

Adaptive signal processing is equivalent to adaptive beam forming processing. That is to say, this adaptive signal processing means processing where processing parameters such as phase, weighting, and so forth, are adaptively changed in accordance with the reception signals, reception signals of desired waves arriving from a target direction of interest or position of interest are selectively extracted, and reception signals of other unwanted waves are suppressed. Particularly, a method called the Capon's method, which is a type of adaptive signal processing, is a method where output (power intensity) as to multiple input signals is minimized, in a state where sensitivity regarding a direction of interest or position of interest is fixed. This is also called the Directionally Constrained Minimization of Power (DCMP) or minimum variance method. Such adaptive signal processing is effective in improving spatial resolution.

One feature of the adaptive signal processing block 007 according to the present embodiment is that difference signals are calculated using reception signals from multiple times (multiple times worth of reception signals obtained by acoustic wave transmission multiple times to the predetermined region), and multiple difference signals are used to perform adaptive signal processing. An example using Capon's method for adaptive signal processing will be described in detail in 3103 in FIG. 2. While Capon's method is used in the present embodiment, other adaptive signal processing, such as MUltiple SIgnal Classification (MUSIC) and Estimation of Signal Parameters via Rotational Invariance Techniques (ESPRIT) may be used.

The flow rate calculating block 008 is a movement information obtaining unit which calculates movement information of an object such as blood flow rate, using output signals output from the adaptive signal processing block 007. In a case of obtaining the blood flow rate for example, the flow rate at multiple positions in the depth direction may be obtained, and not just the blood flow rate at one point (one position) in the subject. Further, the average flow rate or maximum flow rate at a predetermined depth range may be obtained. Moreover, the flow rate at multiple points in time may be obtained in time sequence, so that temporal change of the flow rate can be displayed.

A processing unit is made up of at least the reception circuit system 005, difference processing block 006, adaptive signal processing block 007, and flow rate calculating block 008 in the present embodiment. However, the processing unit according to the present embodiment is not restricted to this, and may include a phasing addition block and an envelope detection processing block (neither illustrated in FIG. 1). A phasing addition block and envelope detection processing block can acquire distribution reflecting difference in acoustic impedance (acoustic property distribution) such as B-mode images and the like, separate from the movement information. Also, B-mode images can be acquired using adaptive signal processing by the device configuration according to a later-described second embodiment, even without a phasing addition block or envelope detection processing block.

The display processing block 009 generates display data using the input movement information, which is output to the display system 010. Specifically, the display processing block 009 processes the input movement information and generates image data to present numerical values representing speed, graphs illustrating temporal change of speed over time, speed distribution representing speed at multiple positions, and so forth. Also, in a case where the processing unit is capable of acquiring acoustic feature distribution as well, image data may be generated to display movement information and acoustic feature distribution together on the same screen, or image data to display the speed distribution obtained as movement information, and the acoustic feature distribution together in a superimposed manner. The display mode may be changed by instructions from the system control unit 004 which has received user input.

Note that in the present embodiment, the difference processing block 006, adaptive signal processing block 007, flow rate calculating block 008, display processing block 009, and system control unit 004 are realized by a processing device such as a central processing unit (CPU), a graphics processing unit (GPU), a field programmable gate array (FPGA) chip, or the like.

The display system 010 is a display device which displays images based on the display data output from the display processing block 009. Examples of the display system 010 include a liquid crystal display (LCD), cathode ray tube (CRT), organic electroluminescence (EL) display, or the like. The display system 010 may be separately provided and connected to the subject information acquiring device, rather than being included in the configuration of the subject information acquiring device according to the present invention.

### Processing Flow at Processing Unit

Next, the processing flow of the processing unit will be described with reference to FIG. 2. FIG. 2 is a flowchart illustrating the processing flow of the present embodiment.

First, in S101, time-sequence reception signals from the reception circuit system 005 are input to the difference processing block 006 by output channels. A signal x_{k,l}[s] input to the difference processing block 006 is the l'th reception signal obtained by the first acoustic wave pulse transmission output from the k'th (k=1, 2, . . . , M) output channel of the reception circuit system 005. Note that the s in brackets means the s'th sample in one time-sequence of reception signals (i.e., one point in the time sequence).

Next, in S102, the difference processing block 006 calculates the difference between two input reception signals (the difference between reception signals obtained from the first pulse and the second pulse of acoustic wave pulses for example), for each output channel, by the following Expression (1), and calculates a difference signal d_{k,l}[s] which is then output.

d_{k,l}[s]=x_{k,l+1}[s]−x_{k,l}[s] Expression (1)

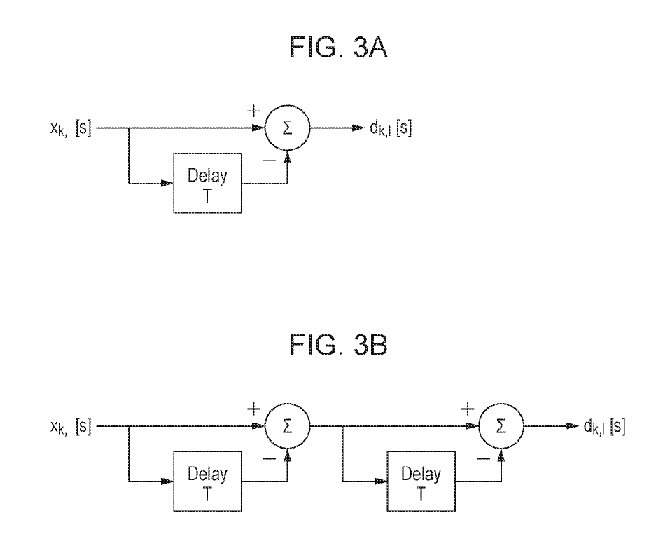

FIG. 3A illustrates a schematic diagram illustrating an example of a difference filter in a case of performing the difference processing at a difference filter. Setting a time equivalent to the transmission interval of acoustic pulses (time interval between l+1'th transmission and 1'th transmission) as a Delay T outputs the difference between x_{k,l+1}[s] and x_{k,l}[s]. The difference processing block 006 extracts multiple difference signals for each output channel by the above processing, and inputs multiple difference signals according to the number of output channels to the adaptive signal processing block 007. In the configuration illustrated in FIG. 3A, at least two difference signals need to be used for each output channel. Note that the present embodiment is not restricted to a difference filter where difference between two reception signals is calculated as in the difference filter illustrated in FIG. 3A, and that a difference filter of a configuration such as illustrated in FIG. 3B may be used instead.

In S103, the adaptive signal processing block 007 performs adaptive signal processing using multiple difference signals input for each channel, of a number corresponding to the output channels. Detailed processing at the adaptive signal processing block 007 will be described now. Note that the present embodiment will be described with regard to a case of having employed the Capon's method as the adaptive signal processing.

First, the adaptive signal processing block 007 uses information of a position of interest (a predetermined position within the subject) instructed from the system control unit 004 to perform delay processing, or phasing processing, so that the phases of the reception signals corresponding to the position of interest are aligned. The signals thus phased are subjected to Hilbert transform. The signals subjected to complex representation will be represented as x′_{k,l}[s′]. In other words, signal x′_{k,l}[s′] is equivalent to the difference signal d_{k,l}[S] corresponding to the k'th output channel which has been subjected to phasing processing and Hilbert transform. The No. s sample input vector X′ₗ[s′] is defined as follows, using the signal x′_{k,l}[s′]. T in Expression (2) indicates a transposed matrix.

X′ₗ[s′]=[x′_{1,l}[s′],x′_{2,l}[s′], . . . ,x′_{M,l}[s′]]^{T} Expression (2)

The adaptive signal processing block 007 further receives input of the difference signal d_{k,l+1}[s] as an input signal. The correlation matrix calculating block 011 within the adaptive signal processing block 007 calculates a correlation matrix R_{xx,l} based on the following Expression (3), using the input vectors X′ₗ[s′] and x′ₗ₊₁[s′]. The input vector x′ₗ₊₁[s′] is the No. s input vector using the signal x′_{k,l+1}[s′]. The signal x′_{k,l+1}[s′] is equivalent to a signal where the difference signal d_{k,l+1}[s] corresponding to the k'th output channel has been subjected to phasing processing and Hilbert transform.

$$Expression ( 3 )$$

$$\begin{matrix}{R}_{xx , l} = E [ \frac{{X}_{l}^{′} [ {s}^{′} ] {X}_{l}^{′ H} [ {s}^{′} ] + {X}_{l + 1}^{′} [ {s}^{′} ] {X}_{l + 1}^{′ H} [ {s}^{′} ]}{C} ] \\ = [ \begin{matrix}\frac{E [ {x}_{1 , l}^{′} [ {s}^{′} ] {x}_{1 , l}^{′ *} [ {s}^{′} ] + {x}_{1 , l + 1}^{′} [ {s}^{′} ] {x}_{1 , l + 1}^{′ *} [ {s}^{′} ] ]}{C} & \frac{E [ {x}_{1 , l}^{′} [ {s}^{′} ] {x}_{2 , l}^{′ *} [ {s}^{′} ] + {x}_{1 , l + 1}^{′} [ {s}^{′} ] {x}_{2 , l + 1}^{′ *} [ {s}^{′} ] ]}{C} & … & \frac{E [ {x}_{1 , l}^{′} [ {s}^{′} ] {x}_{M , l}^{′ *} [ {s}^{′} ] + {x}_{1 , l + 1}^{′} [ {s}^{′} ] {x}_{M , l + 1}^{′ *} [ {s}^{′} ] ]}{C} \\ \frac{E [ {x}_{2 , l}^{′} [ {s}^{′} ] {x}_{1 , l}^{′ *} [ {s}^{′} ] + {x}_{2 , l + 1}^{′} [ {s}^{′} ] {x}_{1 , l + 1}^{′ *} [ {s}^{′} ] ]}{C} & \frac{E [ {x}_{2 , l}^{′} [ {s}^{′} ] {x}_{2 , l}^{′ *} [ {s}^{′} ] + {x}_{2 , l + 1}^{′} [ {s}^{′} ] {x}_{2 , l + 1}^{′ *} [ {s}^{′} ] ]}{C} & … & \frac{E [ {x}_{2 , l}^{′} [ {s}^{′} ] {x}_{M , l}^{′ *} [ {s}^{′} ] + {x}_{2 , l + 1}^{′} [ {s}^{′} ] {x}_{M , l + 1}^{′ *} [ {s}^{′} ] ]}{C} \\ ⋮ & ⋮ & ⋱ & ⋮ \\ \frac{E [ {x}_{M , l}^{′} [ {s}^{′} ] {x}_{1 , l}^{′ *} [ {s}^{′} ] + {x}_{M , l + 1}^{′} [ {s}^{′} ] {x}_{1 , l + 1}^{′ *} [ {s}^{′} ] ]}{C} & \frac{E [ {x}_{M , l}^{′} [ {s}^{′} ] {x}_{2 , l}^{′ *} [ {s}^{′} ] + {x}_{M , l + 1}^{′} [ {s}^{′} ] {x}_{2 , l + 1}^{′ *} [ {s}^{′} ] ]}{C} & … & \frac{E [ {x}_{M , l}^{′} [ {s}^{′} ] {x}_{M , l}^{′ *} [ {s}^{′} ] + {x}_{M , l + 1}^{′} [ {s}^{′} ] {x}_{M , l + 1}^{′ *} [ {s}^{′} ] ]}{C}\end{matrix} ]\end{matrix}$$

The exponent H in Expression (3) represents the complex conjugate transpose, and the exponent asterisk * represents the complex conjugate. C represents the number of differential signals being used (two in this case), and serves as a divider to average the correlation matrices based on the multiple difference signals. E[·] represents processing to calculate the time average, this meaning to change the order of the sample No. (s′ in this case) to calculate the average at multiple sample points.

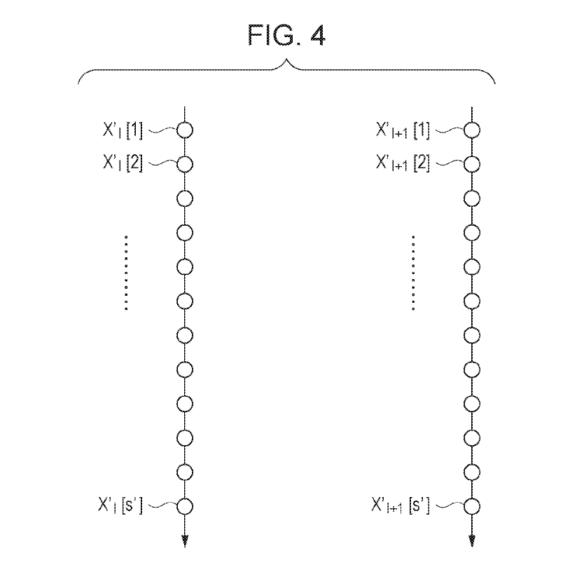

That is to say, the above expression (3) represents calculating the final correlation matrix R_{xx,l} by averaging the correlation matrices based on each of the multiple difference signals, and averaging in the temporal direction. This will be described in detail with reference to FIG. 4. Note that the term “averaging in the temporal direction” means to calculate the average in the distance direction within an observation region.

FIG. 4 is a schematic diagram illustrating X′ₗ[s′] and X′ₗ₊₁[s′]. Here, “averaging correlation matrices based on each of multiple difference signals” means that the correlation matrices are averaged based on each of input vectors indicating the difference signals. For example, averaging of a correlation matrix (X′ₗ[s′]·X′ₗ^{H}[s′]) based on an input vector X′ₗ[s′] and a correlation matrix (X′ₗ₊₁[s′]·X′ₗ₊₁^{H}[s′]) based on an input vector X′ₗ₊₁ [s′] is given.

In a case of averaging correlation matrices with multiple difference signals, the correlation matrices are preferably averaged based on each of sample points corresponding to a predetermined position within the subject (i.e., sample points corresponding to the same position), as shown in Expression (3). For example, averaging of a correlation matrix (X′ₗ[1]·X′ₗ^{H}/[1]) based on an input vector X′ₗ[1] and a correlation matrix (X′ₗ₊₁[1]·X′ₗ₊₁^{H}[1]) based on an input vector X′ₗ₊₁ [1] is given. Note however, that the term “same position” is not restricted to being the same position in the strictest sense, and includes a range regarding which there is no problem in performing processing deeming to be the same region.

Expression (3) also includes averaging the correlation matrices in the temporal direction, in addition to averaging the correlation matrices among the difference signals. For example, in a case of correlation matrices averaged among the difference signals being further averaged in the temporal direction, the value of s′ in the correlation matrix after averaging is varied. The correlation matrices after averaging are averaged at difference sample points (e.g., s′=1, s′=2). Note that the temporal direction in this case corresponds to the direction of travel of acoustic waves (ultrasound beam) transmitted from the probe 001, and typically corresponds to the distance direction (depth direction) within the subject.

In practice, difference signals are sequentially input to the adaptive signal processing block 007. Accordingly, the correlation matrixes based on each sample point in one difference signal input prior are preferably averaged in the temporal direction beforehand. Thereafter, the averaged correlation matrices are averaged among the multiple difference signals. For example, a correlation matrix (X′ₗ[1]·X′ₗ^{H}[1]) based on an input vector X′ₗ[1] and a correlation matrix (X′ₗ[2]·X′ₗ^{H}[2]) based on an input vector X′ₗ[2] are averaged beforehand. Next, a correlation matrix (X′ₗ₊₁[1]·X′ₗ₊₁^{H}[1]) based on an input vector X′ₗ₊₁[1] and a correlation matrix (X′ₗ₊₁[2]·X′ₗ₊₁^{H}[2]) based on an input vector X′ₗ₊₁[2] are also averaged. Finally, the correlation matrices after averaging are averaged among the difference signals.

Alternatively, the adaptive signal processing block 007 may perform averaging in the temporal direction and averaging among difference signals at the same time. In this case, multiple difference signals which are sequentially output from the difference processing block 006 may be stored in memory.

Averaging of the correlation matrices in the temporal direction is not indispensable in the present embodiment. Due to averaging being performed of the correlation matrices among difference signals using these multiple difference signals, accuracy of the correlation matrix output in the end improves. In a case of performing averaging in the temporal direction, the width of the temporal average is preferably no higher than the resolution at a region of a predetermined depth, and this may be changed for each region in the depth direction.

Also, in practice regarding the correlation matrix averaging processing, the components within the correlation matrices may be averaged. There are cases therein where it is permissible that not all components within the correlation matrix output at the end are obtained. Accordingly, this “averaging of correlation matrices” includes cases of only averaging a part of the components.

Averaging of the correlation matrices may be performed by simply integrating (adding) the correlation matrices. That is to say, C=1 may hold regardless of the number of difference signals in Expression (3). In the present invention, integration processing is to be understood to include averaging.

Also, while using the Capon's method without performing spatial averaging has been described, the Capon's method may be used with spatial averaging.

Next, the adaptive signal processing block 007 obtains a weight vector W under the conditions of the following Expression (4), using the correlation matrix output from the correlation matrix calculating block 011.

$${min}_{W} ( {W}^{H} {R}_{xx , l} W ) \\ subject to {W}^{H} a = 1 \qquad Expression ( 4 )$$

These conditions mean that output (power intensity) is minimized in a state where sensitivity in a desired direction (direction of interest) is constrained to 1. In the Expression (4), “a” represents a steering vector, stipulating the desired direction which is the direction of interest. Calculating the optimal weight Wopt from such conditions yields the following Expression (5).

$$Wopt = \frac{{( {R}_{xx , l} + η E )}^{- 1} a}{{{a}^{H} ( {R}_{xx , l} + η E )}^{- 1} a} \qquad Expression ( 5 )$$

Note that ηE has been added to stabilize inverse matrix calculation, where η is a constant or a value which changes in accordance with the value of R_{xx,l} or the like, and E is an identity matrix. Using this optimal weight enables the output power to be minimized in a state where the sensitivity in the desired direction is 1. Also, using this optimal weight enables formation of a reception pattern having directivity, where the sensitivity in the desired direction is 1, and sensitivity is low regarding directions from which noise components travel. The adaptive signal processing block 007 then multiples the input vector X1[s′] by this optimal weight Wopt to obtain an output signal yₗ[s′] after adaptive signal processing.

yₗ[s′]=W^{H}optXₗ′[s′] Expression (6)

In S104, the flow rate calculating block 008 uses the output signal yₗ[s′] from the adaptive signal processing block 007 to calculate the flow rate v_{c}, which is the movement information, from Expression (7).

$$\begin{matrix}{v}_{c} = \frac{{λ}_{c} \overline{{θ}_{c} [ {s}^{′} ]}}{4 π T} \\ = \frac{{λ}_{c} arg [ {\int }_{{S}_{obs} - {S}_{ave} / 2}^{{S}_{obs} + {S}_{ave} / 2} {{y}_{l} [ {s}^{′} ]}^{*} {y}_{l + 1} [ {s}^{′} ] d {s}^{′} ]}{4 π T}\end{matrix} \qquad Expression ( 7 )$$

In Expression (7), S_{obs} represents a sample No. corresponding to the observation depth (predetermined position in the depth direction), Sₐᵥₑ represents the temporal average width, and T represents the repetition cycle of transmitting the acoustic wave pulses (time interval between l+1'th transmission and l'th transmission). λ_{c} represents the wavelength of the transmission frequency, and θ_{c} represents the phase difference between yₗ and yₗ₊₁. Calculating and using the phase difference (change in phase) among the difference signals obtained by transmitting the acoustic waves multiple times enables the speed of movement of the object to be obtained. Displacement can also be obtained by multiplying this speed of movement by the repetition cycle.

The flow rate v_{c} calculated in this way is input to the display processing block 009. The display processing block 009 constructs image data using the input flow rate as described above, and outputs to the display system 010.

An example of an image displayed on the display system 010 will be described with reference to FIG. 5. FIG. 5 illustrates the way in which temporal change of the flow rate at three types of depth ranges (5 to 15 mm, 15 to 25 mm, and 25 to 35 mm). The average flow rate and maximum flow rate are also illustrated in numerical values for each depth range.

### Advantages of Present Embodiment

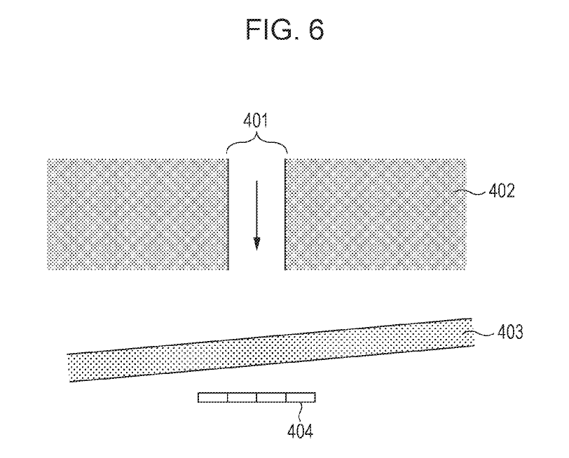

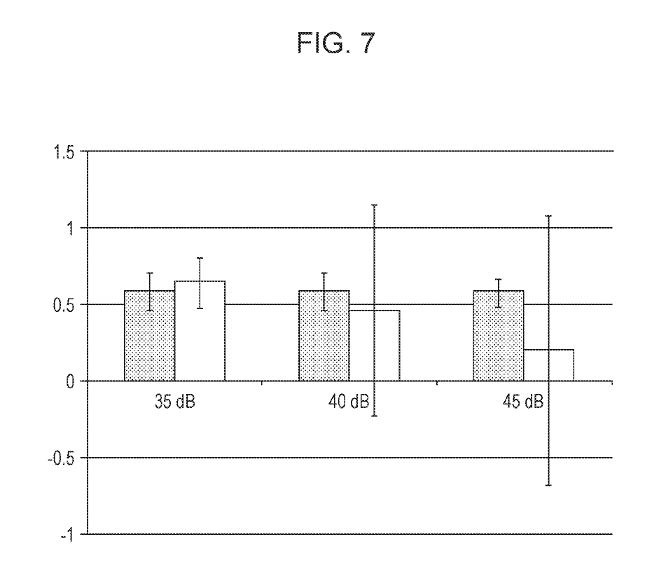

Next, the advantages of the present embodiment will be described with reference to FIGS. 6 and 7. FIG. 6 is a simplified model of an organism. In this model, the blood in the region 401 flows in the direction indicated by the arrow, with surrounding tissue 402 situated therearound. Further, a strong reflector 403 assuming a skull bone is situated on the path where ultrasound waves, or acoustic waves, are transmitted and received. Ultrasound waves are transmitted using four conversion elements 404, and reflected waves are obtained from around the region 401. Signals obtained by imparting fluctuation which changes over time to the obtained reception signals are taken as the input signals for the processing of the present embodiment. These fluctuations assume body motion such as pulse, shaking of the hands of the user operating the probe, and so forth. The results of simulation are illustrated in FIG. 7.

FIG. 7 illustrates calculation results of flow rate (vertical axis) in three cases, where the intensity ratios of reflected waveforms from the surrounding tissue 402 as to the reflected waveforms from the region 401 are 35 dB, 40 dB, and 45 dB. At each intensity ratio, the processing results of using only an MTI filter (only difference processing) is the bar to the right, and the processing results using the present embodiment is the bar to the left. Note that in the model, the blood flow is set to pass near the probe at a flow rate of +0.5 m/sec, so 0.5 m/sec is the true value of the flow rate.

It can be seen from the processing results using the MTI filter alone that the estimation accuracy of the flow speed drops when the intensity ration exceeds 40 db, and that the processing results using the present embodiment show that the flow rate is being accurately estimated even if the intensity ratio exceeds 45 dB. The error bars indicate the variance in cases where simulation conditions where changed. Thus, the speed can be accurately obtained in the present embodiment even if a great clutter component exists.

Next, the reason why the advantages of the present embodiment can be obtained will be described. First, signals indicating temporal fluctuation component among reception signals are extracted in the difference processing according to the present embodiment. The temporal fluctuation component is equivalent to the component based on reflection waves from a reflector moving within the subject (moving component). That is to say, the difference processing acts to weaken (ideally to eliminate) reception signals due to reflected waves from reflectors not moving during the repetition cycle T which is the transmission interval of acoustic pulses. However, while difference processing removes signals from reflectors which do not move during the repetitive cycle T, signals of clutter components from moving reflectors (clutter signals) may not be removed in some cases. The results of using only the MTI filter in FIG. 7 represents a case of residual clutter signals that were not completely removed affecting flow rate estimation.

In the present embodiment, using adaptive signal processing enables clutter components arriving from directions other than the direction of interest (desired direction) to be suppressed, even if clutter signals which were not completely removed in such difference processing are included. That is to say, clutter signals due to reflected waves from moving reflectors other than the object can be reduced, and signals of reflected waves from the object can be selectively acquired, so flow rate estimation can be performed with higher accuracy.

Also, performing difference processing in the present embodiment ideally removes all components not temporally fluctuating between reception signals (temporal non-fluctuation components), and components temporally fluctuating between reception signals (temporal fluctuation components) are primarily extracted. However in practice, there are cases where temporal non-fluctuation components cannot be completely removed in a case of using a difference filter such as an MTI filter or the like in the difference processing block 006. There are also cases where part of the temporal fluctuation components may be removed by the difference filter is the temporal fluctuation is small (e.g., low-frequency components where the movement is slow).

Accordingly, the phrase “extract signals indicating temporal fluctuation component” in the present invention encompasses not only cases of extracting all temporal fluctuation components, but also cases of extracting part of temporal fluctuation components. Some temporal non-fluctuation components may also be included besides the temporal fluctuation components. Even if performing difference processing does not remove all temporal non-fluctuation components, this arrangement is advantageous if temporal non-fluctuation components are reduced as compared with a case of not performing difference processing.

The “signals indicating temporal fluctuation component” that have been thus extracted, are signals suitable for adaptive signal processing. The basic operation of adaptive signal processing is to suppress clatter component from a particular direction. In the present embodiment, the extracted “signals indicating temporal fluctuation component among reception signals” have the temporal non-fluctuation component reduced, and accordingly power due to the clutter component has been suppressed beforehand. Accordingly, using such “signals indicating temporal fluctuation component” to perform adaptive signal processing causes the adaptive signal processing to work effectively. In other words, the power due to the clutter components of direction which was not completely suppressed can be suppressed by the adaptive signal processing, and the temporal fluctuation component of the object can be calculated (i.e., the temporal fluctuation component of the clutter signals can be suppressed).

Note that the “movement information” of the object within the subject refers to information relating to “speed” and “displacement”. Specific examples include blood flow rate (movement speed of a scatterer group formed of red blood cells, or the like), displacement of tissue, and the like. Speed and displacement may be obtained as speed distribution and displacement distribution.

One feature of the present embodiment is in the calculation method of the correlation matrix at the adaptive signal processing block 007, as described when describing Expression (3). The adaptive signal processing block 007 performs adaptive signal processing using multiple difference signals calculating using reception signals from multiple times (multiple times worth of reception signals, obtained by multiple times of acoustic wave transmission to the same region). That is to say, multiple difference signals are input to the adaptive signal processing block 007 for each channel, and a single signal (signal after adaptive signal processing) is output.

Thus, executing adaptive signal processing using phase matrices based on each of multiple input signals (in the present embodiment, equivalent to multiple difference signals) results in improved accuracy of correlation matrices as compared to a case of performing adaptive signal processing using correlation matrices based on one input signal. Accordingly, output signals with higher accuracy can be obtained.

Further, accuracy of correlation matrices is improved in the present embodiment as compared with a case of averaging correlation matrices based on multiple sample points in one input signal, in the temporal direction alone, as well. The reason is that by averaging correlation matrices based on multiple sample points in each of multiple input signals, among the input signals, the width of the depth direction used for averaging can be narrowed. Accordingly, even if there is change in the distribution of the scatterer in the width used for averaging, the correlation matrix can be calculated accurately due to the width being narrower.

### Second Embodiment

Next, a second embodiment will be described. The subject information acquiring device according to the first embodiment acquires flow rate using output from adaptive signal processing; the subject information acquiring device according to the present embodiment acquires acoustic property distribution such as B-mode images and the like, using output from adaptive signal processing.

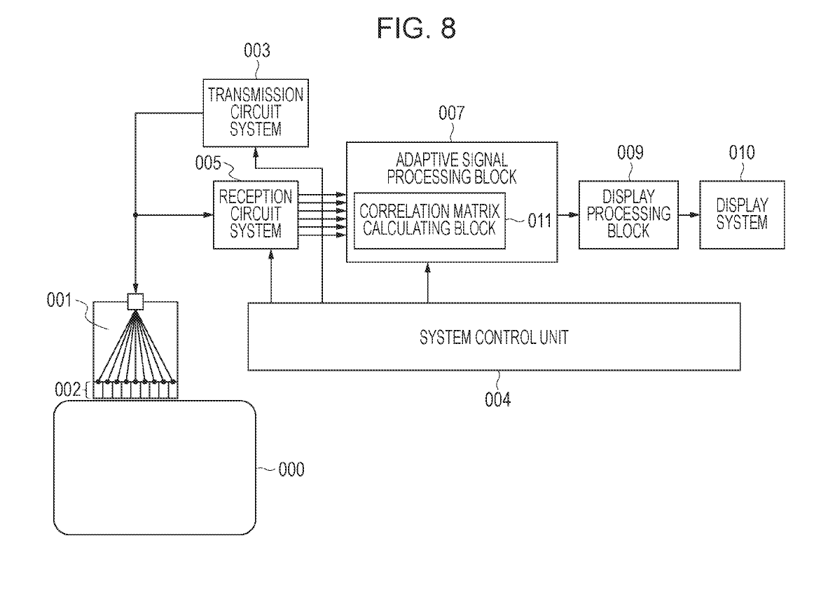

FIG. 8 is a schematic diagram illustrating the configuration of the subject information acquiring device according to the present embodiment. The subject information acquiring device according to the present embodiment includes the probe 001 having multiple conversion elements 002, the reception circuit system 005, the transmission circuit system 003, the adaptive signal processing block 007, the system control unit 004, the display processing block 009, and the display system 010.

This configuration is the same as that in FIG. 1 except for the difference processing block 006 and flow rate calculating block 008 having been omitted, so detailed description of common portions will be omitted. In the same way with the processing flow, up to transmission of acoustic waves from the probe 001 to a predetermined region multiple times, and obtaining reception signals, is the same as that in the first embodiment, so description will start after that point.

The adaptive signal processing block 007 receives, from the reception circuit system 005, multiple times worth of reception signals obtained by multiple times of acoustic wave transmission, for as many output channels there are. The adaptive signal processing block 007 uses information of a position of interest instructed from the system control unit 004 to perform delay processing, or phasing processing, so that the phases of the reception signals corresponding to the position of interest are aligned. The reception signals thus phased are subjected to Hilbert transform.

In the present embodiment, the signal after the Hilbert transform is equivalent to the input signal x′_{k,l}[s′] in Expression (2) in the first embodiment. The No. s′ sample input vector is represented by X′ₗ[s′], using the input signal x′_{k,l}[s′]. The correlation matrix is calculated based on Expression (3) using this input vector. The adaptive signal processing including calculation of the correlation matrix is the same as with the first embodiment up to obtaining the output signal yₗ[s′] based on the input signal x′_{k,l}[s′]. That is to say, adaptive signal processing is performed using correlation matrices based on each of multiple input signals (equivalent to multiple reception signals in the present embodiment).

Thereafter, the adaptive signal processing block 007 calculates the envelope of the calculated output signal yₗ[s′], and outputs to the display processing block 009. Note that the power may be calculated in the adaptive signal processing using the following expression.

$${P}_{min} = \frac{1}{2} \frac{1}{{{a}^{H} ( Rxx , l + η E )}^{- 1} a}$$

The display processing block 009 uses the input signals to construct image data representing acoustic property distribution, and outputs to the display system 010.

The acoustic property distribution image thus acquired has reduced noise from clutter components, and deterioration of image quality is suppressed. The reason why this advantage is obtained will be described. Note that in the following description, the advantage in that noise due to clutter component is reduced and an accurate correlation matrix is calculated, is the same as with the first embodiment.

In Expression (3), the width for averaging correlation matrices in the temporal direction (i.e., the width in the depth direction) is the width for calculating the correlation between the reflection wave component from the desired direction, and clutter component from scatterers existing in other directions. Accordingly, in a case where the distribution of scatterers which cause the clutter component within that width changes, a correlation matrix including the amount of change is also calculated. Particularly, in a case where the distribution of scatterers is changing greatly within that width, the results of adaptive signal processing using the calculated correlation matrix may not be able to sufficiently suppress the clutter component. Also, in a case where the width in the depth direction for averaging is wide, there are many directions in which scatterers exist, meaning that there are many directions in which suppression by adaptive signal processing has to be performed.

However, simply narrowing the width for averaging results in the calculated correlation matrix being easily affected by noise component, and it is even more likely that the effects of suppressing clutter component cannot be sufficiently exhibited.

Accordingly, correlation matrices are calculated using Expression (3) in the first and second embodiments. That is to say, correlation matrices are averaged based on each of multiple input signals. This allows the width for averaging to be narrowed, and even if there is change in the distribution of scatterers causing clutter component, the final correlation matrix can be calculated with high accuracy.

Accordingly, the present embodiment is highly effective in suppressing clutter component by adaptive signal processing, due to performing the adaptive signal processing using highly accurate correlation matrices. Acoustic property distribution is acquired using output signals from the adaptive signal processing block 007 such as described above, so the image quality of the obtained image is improved. Spatial resolution and contrast may be improved in particular.

### Third Embodiment

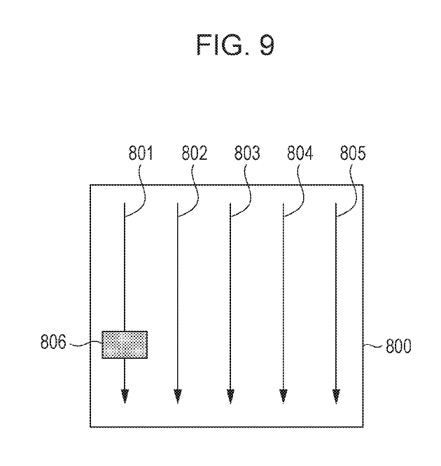

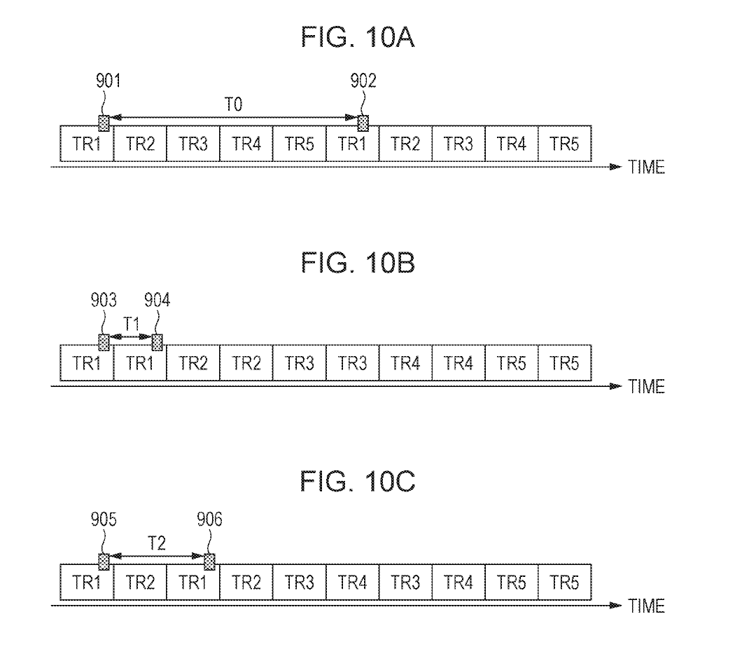

A third embodiment will be described with reference to FIGS. 9 and 10. A feature of the present embodiment is transmission control of acoustic waves. In the following description, acoustic property distribution is obtained in the same way as with the subject information acquiring device according to the second embodiment, so description of the device will be omitted, and the transmission control by the system control unit 004 will be described in detail.

FIG. 9 is a schematic diagram illustration a region 800 where an image of acoustic property distribution is obtained. Acoustic waves are transmitted from the probe 001 along five scanning lines of scanning line 801 through scanning line 805 within the region 800, thus yielding one frame worth of image corresponding to the region 800. The term scanning line as used here refers to a virtual line on the direction of travel of acoustic waves transmitted from the probe 001 (equivalent to the direction of acoustic wave beams).

FIGS. 10A through 10C are schematic diagrams illustrating transmission/reception timing of the scanning lines 801 through 805. TR1 in FIG. 10 represents the transmission/reception timing of the scanning line 801. In the same way, TR2 represents the transmission/reception timing of the scanning line 802, TR3 scanning line 803, TR4 scanning line 804, and TR5 scanning line 805.

First, FIG. 10A illustrates an example of transmitting acoustic waves in order from scanning line 801 to scanning line 805, one time each. Attention is to be given to a predetermined region 806 within region 800 at this time. In this case, the reception signals each obtained at time width 901 and time width 902 each correspond to the acoustic waves from the region 806. The adaptive signal processing block 007 calculates correlation matrices using these reception signals. In this case, the temporal difference between time width 901 and time width 902 is T0.

Next, FIG. 10B will be described. In FIG. 10B, the region corresponding to the scanning line 801 within the region 800 is first scanned by acoustic waves along the scanning line 801 by transmitting two times consecutively. Thereafter, transmission/reception is also perform on scanning line 802 two times consecutively. In the same way, control is effected such that transmission/reception is performed on the scanning lines 803, 804, and 805 each twice along the same scanning line before going to the next scanning line. In this case, the reception signals each obtained at time width 903 and time width 904 each correspond to the acoustic waves from the region 806. Correlation matrices are calculated using these reception signals. In this case, the temporal difference between time width 903 and time width 904 is T1.

Comparing the time difference T0 in FIG. 10A and the time difference T1 in FIG. 10B, T1<T0 holds. That is to say, the time difference in the timing for obtaining the reception signals of the same region 806 is smaller in the transmission control illustrated in FIG. 10B. Accordingly, the arrangement illustrated in FIG. 10B where the transmission is consecutively performed to the same region multiple times before going to the next region is preferable.

The reason is that, when handling tissue which moves quickly, the smaller the time difference between reception signals (time interval), the smaller the amount of movement of the tissue within the time interval is. The smaller the amount of movement is, the smaller the difference between reception signals is, thus improving the accuracy of averaged correlation matrices. Accordingly, the image quality of the obtained acoustic property distribution also improves.

Alternatively, the present embodiment may perform transmission control as illustrated in FIG. 10C. In FIG. 10C, acoustic wave transmission is performed once each in the order of scanning line 801, scanning line 802, scanning line 801, scanning line 802, followed by acoustic wave transmission being performed in the order of scanning line 803, scanning line 804, and scanning line 803, scanning line 804. In this case, the reception signals each obtained at time width 905 and time width 906 each correspond to the acoustic waves from the region 806. The temporal difference between time width 905 and time width 906 is T2. Accordingly, the time difference T2 in this case is also smaller than the time difference T0 in FIG. 10A, so the accuracy of correlation matrices improves over that of the case in FIG. 10A.

That is to say, in a case of the display processing block 009 acquiring one frame worth of acoustic property distribution corresponding to the region 800, transmission of acoustic waves is preferably performed to the predetermined region multiple times, before the probe 001 ends transmission of acoustic waves to all regions within the region 800. Transmission/reception control of the probe 001 is performed by the system control unit 004.

Also, the present embodiment is not restricted to generating B-mode images, and may be used to generate a Doppler image by obtaining motion information of the object as with the first embodiment. Moreover, an arrangement may be made where the entire image of the region 800 is generated as a B-mode image, with just the predetermined region 806 being acquired as motion information of the object.

### Fourth Embodiment

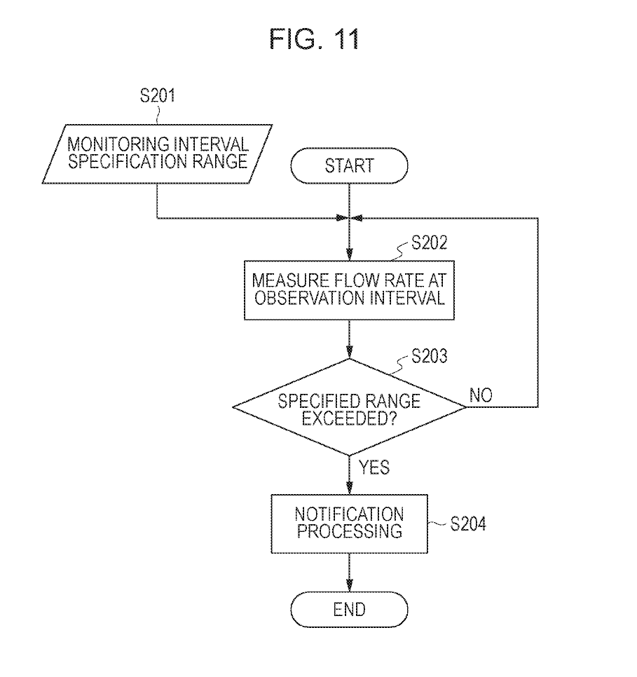

Next, a fourth embodiment will be described with reference to FIG. 11. A feature of the present embodiment is to perform monitoring of the flow rate of the object at predetermined time intervals. In the following description, the movement information of the object is acquired using a subject information acquiring device the same as that in the first embodiment, so description of the device will be omitted.

FIG. 11 is a flowchart for describing processing to monitor the flow rate of an object in the present embodiment.

First, a user sets a monitoring interval (e.g., once every second) and a specification range (e.g., plus-minus 0.1 m/sec) to the system control unit 004 (S201), using an input device (a mouse or touch panel, or buttons, dials and the like on the device) omitted from illustrating in the drawings.

The flow rate calculating block 008 performs measurement of the flow rate at the set monitoring interval (S202). This flow rate measurement can be accurately carried out by using the method according to the first embodiment.

Next, in S203, if the measurement result of the flow rate has not exceeded the set specification range, the flow returns to S202, and if the set specification range has been exceeded, the flow advances to S204.

In S204, the system control unit 004 notifies the user by way of a predetermined notification method. This notification method may be a display on the display system 010, or an audible notification. The flow may return to the start and resume flow measurement after having performed the notification processing.

Accordingly, in the present embodiment, the flow rate of the object is obtained at predetermined time intervals. The system control unit 004 determines whether or not the flow rate has exceeded a predetermined range or a predetermined value, and notifies information based on the determination result. For example, in a case of having injected an anticoagulant into a blood vessel, the user may be able to judge whether the anticoagulant has reached the object region by being able to tell whether the blood flow rate has become faster than a predetermined value.

The monitoring interval and specification range may be preset to the device rather than being input by the user.

Also, in the present embodiment, the display processing block 009 which has received instructions from the system control unit 004 may display flow rate values on the display system 010 at each flow rate measurement in S202. Further, each flow rate value may be displayed on a graph so that the change of flow rate values over time can be comprehended.

According to the present embodiment, deterioration in acquisition accuracy of motion information and deterioration in image quality of acoustic property distribution can be suppressed, even in cases where unnecessary reflection waves are present.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-150761, filed Jul. 19, 2013, which is hereby incorporated by reference herein in its entirety.

### REFERENCE SIGNS LIST

000 Subject
001 Probe
002 Conversion element
003 Transmission circuit system
004 System control unit
005 Reception circuit system
006 Difference processing block
007 Adaptive signal processing block
008 Flow rate calculating block
009 Display processing block
010 Display system

1. A subject information acquiring device, comprising:
a plurality of conversion elements, each configured to transmit acoustic waves as to a subject, receive reflected waves which have reflected within the subject, and convert the received reflected waves into time-sequence reception signals; and
a processing unit configured to acquire information of within the subject by performing adaptive beam forming processing using a plurality of the reception signals,
wherein, to output a plurality of times worth of reception signals based on each of a plurality of times of acoustic wave transmission, at least a part of the plurality of conversion elements perform acoustic wave transmission as to a predetermined region within the subject the plurality of times, and
wherein, in the adaptive beam forming processing, the processing unit performs integration processing of a plurality of correlation matrices obtained using the plurality of times worth of reception signals.

2. The subject information acquiring device according to claim 1, wherein, in the adaptive beam forming processing, the processing unit performs integration processing of correlation matrices based on each of a plurality of sample points, corresponding to a predetermined position within the subject, using the plurality of times worth of reception signals.

3. The subject information acquiring device according to claim 2, wherein, in the adaptive beam forming processing, the processing unit further performs integration processing of correlation matrices based on each of a plurality of sample points, corresponding to different positions within the subject.

4. The subject information acquiring device according to claim 1, wherein, in a case where the conversion elements perform acoustic wave transmission to a predetermined region corresponding to one frame worth of acoustic property distribution, in order for the processing unit to acquire the one frame worth of acoustic property distribution including the region, the at least part of the conversion elements perform acoustic wave transmission to the predetermined region multiple times, before the acoustic wave transmission to the region corresponding to the one frame worth by the plurality of conversion elements ends.

5. The subject information acquiring device according to claim 1, wherein the processing unit further includes
an extracting unit configured to extract, using the plurality of times worth of reception signals, a plurality of signals indicating temporal fluctuation component among reception signals,
an adaptive signal processing unit configured to perform adaptive beam forming using a plurality of the signals indicating temporal fluctuation component, and
a motion information acquisition unit configured to acquire, using output signals from the adaptive signal processing unit, motion information of an object;
wherein the adaptive signal processing unit performs integration processing of correlation matrices based on each of the plurality of the signals indicating temporal fluctuation component.

6. The subject information acquiring device according to claim 5,
wherein the extracting unit extracts a plurality of the signals indicating temporal fluctuation component for each output channel from the conversion elements, and
wherein the adaptive signal processing unit performs adaptive beam forming processing using the output channels worth of the plurality of the signals indicating temporal fluctuation component, for each output channel.

7. The subject information acquiring device according to claim 5, wherein the extracting unit extracts the signals indicating temporal fluctuation component by an MTI filter.

8. The subject information acquiring device according to claim 5, wherein the processing unit acquires motion information of the object at predetermined time intervals.

9. The subject information acquiring device according to claim 1, wherein the processing unit further includes an adaptive signal processing unit configured to perform adaptive beam forming processing using the plurality of times worth of reception signals,
wherein the adaptive signal processing unit performs integration processing of correlation matrices based on each of the plurality of times worth of reception signals.

10. The subject information acquiring device according to claim 9, wherein the adaptive signal processing unit performs adaptive beam forming processing using the plurality of reception signals output at each of output channels from the conversion elements.

11. The subject information acquiring device according to claim 1, wherein the processing unit performs the adaptive beam forming processing such that power is minimized in a state where sensitivity as to a direction of interest is fixed.

12. A subject information acquiring device, comprising:
a plurality of conversion elements, each configured to transmit acoustic waves as to a subject, receive reflected waves which have reflected within the subject, and convert the received reflected waves into time-sequence reception signals; and
a processing unit including an adaptive signal processing unit configured to perform adaptive beam forming processing using the plurality of reception signals, wherein the processing unit is configured to acquire information of within the subject using output signals from the adaptive signal processing unit;
wherein, to output a plurality of times worth of reception signals based on each of a plurality of times of acoustic wave transmission, at least a part of the plurality of conversion elements perform acoustic wave transmission as to a predetermined region within the subject the plurality of times, and
wherein, to obtain one output signal as to the plurality of input signals, the adaptive signal processing unit performs the adaptive beam forming processing using a plurality of input signals based on the plurality of times worth of reception signals.

13. A subject information acquiring method for a subject information acquiring device having a plurality of conversion elements, each configured to transmit acoustic waves as to a subject, receive reflected waves which have reflected within the subject, and convert the received reflected waves into time-sequence reception signals, the subject information acquiring method comprising:
acquiring information of within the subject by performing adaptive beam forming processing using a plurality of the reception signals,
wherein, to output a plurality of times worth of reception signals based on each of a plurality of times of acoustic wave transmission, performing, via at least a part of the plurality of conversion elements, acoustic wave transmission as to a predetermined region within the subject the plurality of times, and
wherein, in the adaptive beam forming processing, integration processing is performed of a plurality of correlation matrices obtained using the plurality of times worth of reception signals.

14. The subject information acquiring method according to claim 13, wherein, in the adaptive beam forming processing, integration processing is performed of correlation matrices based on each of a plurality of sample points, corresponding to a predetermined position within the subject, using the plurality of times worth of reception signals.

15. The subject information acquiring method according to claim 14, wherein, in the adaptive beam forming processing, integration processing of correlation matrices further is performed based on each of a plurality of sample points, corresponding to different positions within the subject.

16. The subject information acquiring method according to claim 13, wherein, in a case where the conversion elements perform acoustic wave transmission to a predetermined region corresponding to one frame worth of acoustic property distribution, in order to acquire the one frame worth of acoustic property distribution including the region, the at least part of the conversion elements perform acoustic wave transmission to the predetermined region multiple times, before the acoustic wave transmission to the region corresponding to the one frame worth by the plurality of conversion elements ends.

17. The subject information acquiring method according to claim 13, further comprising:
extracting, using the plurality of times worth of reception signals, a plurality of signals indicating temporal fluctuation component among reception signals,
performing adaptive beam forming using a plurality of the signals indicating temporal fluctuation component, and
acquiring, using output signals from performing adaptive beam forming, motion information of an object;
wherein performing adaptive beam forming includes performing integration processing of correlation matrices based on each of the plurality of the signals indicating temporal fluctuation component.

18. The subject information acquiring method according to claim 17,
wherein extracting includes extracting a plurality of the signals indicating temporal fluctuation component for each output channel from the conversion elements, and
wherein performing adaptive beam forming includes using the output channels worth of the plurality of the signals indicating temporal fluctuation component, for each output channel.

19. The subject information acquiring method according to claim 17, wherein acquiring includes acquiring motion information of the object at predetermined time intervals.

20. The subject information method device according to claim 13,
wherein acquiring further includes performing adaptive beam forming processing; using the plurality of times worth of reception signals, and
wherein performing adaptive beam forming processing includes performing integration processing of correlation matrices based on each of the plurality of times worth of reception signals.

21. The subject information acquiring method according to claim 20, performing adaptive beam forming processing includes using the plurality of reception signals output at each of output channels from the conversion elements.

22. The subject information acquiring method according to claim 13, wherein acquiring includes performing the adaptive beam forming processing such that power is minimized in a state where sensitivity as to a direction of interest is fixed.

23. A subject information acquiring method for a subject information acquiring device having a plurality of conversion elements, each configured to transmit acoustic waves as to a subject, receive reflected waves which have reflected within the subject, and convert the received reflected waves into time-sequence reception signals, the subject information acquiring method comprising:
performing adaptive beam forming processing using the plurality of reception signals, wherein performing includes acquiring information of within the subject using output signals from the adaptive beam forming processing;
wherein, to output a plurality of times worth of reception signals based on each of a plurality of times of acoustic wave transmission, at least a part of the plurality of conversion elements perform acoustic wave transmission as to a predetermined region within the subject the plurality of times, and
wherein, to obtain one output signal as to the plurality of input signals, performing adaptive beam forming processing includes using a plurality of input signals based on the plurality of times worth of reception signals.

24. A program to cause a computer to perform the steps of the subject information acquiring method according to claim 13.
